SEQUENCE LISTING (36 sequences)

SEQ ID NO: 1 - protein, 7 aa, Artificial Sequence
  heavy chain variable region (VH) complementarity determining region (CDR), CDR1
GYAFSSS

SEQ ID NO: 2 - protein, 6 aa, Artificial Sequence
  VH CDR, CDR2
YPGDED

SEQ ID NO: 3 - protein, 10 aa, Artificial Sequence
  VH CDR, CDR3
SLLYGDYLDY

SEQ ID NO: 4 - protein, 10 aa, Artificial Sequence
  light chain variable region (VL) CDR, CDR1
SASSSVSYMH

SEQ ID NO: 5 - protein, 7 aa, Artificial Sequence
  VL CDR, CDR2
DTSKLAS

SEQ ID NO: 6 - protein, 9 aa, Artificial Sequence
  VL CDR, CDR3
QQWNINPLT

SEQ ID NO: 7 - protein, 119 aa, Artificial Sequence
  VH domain sequence
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSS

SEQ ID NO: 8 - protein, 107 aa, Artificial Sequence
  VL domain sequence
QIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 9 - skipped

SEQ ID NO: 10 - protein, 5 aa, Artificial Sequence
  VH CDR, CDR1
NFAMA

SEQ ID NO: 11 - protein, 17 aa, Artificial Sequence
  VH CDR, CDR2
SISTGGGNTYYRDSVKG

SEQ ID NO: 12 - protein, 18 aa, Artificial Sequence
  VH CDR, CDR3
QRNYYDGSYDYEGYTMDA

SEQ ID NO: 13 - protein, 11 aa, Artificial Sequence
  VL CDR, CDR1
RSSQDIGNYLT

SEQ ID NO: 14 - protein, 7 aa, Artificial Sequence
  VL CDR, CDR2
GAIKLED

SEQ ID NO: 15 - protein, 7 aa, Artificial Sequence
  VL CDR, CDR3
LQSIQYP

SEQ ID NO: 16 - protein, 127 aa, Artificial Sequence
  VH domain sequence
EVQLVESGGGLVQPGRSLKLSCAASGFTFSNFAMAWVRQPPTKGLEWVASISTGGGNTYYRDSVKGRFTISRDDAKNTQYLQMDSLRSEDTATYYCARQRNYYDGSYDYEGYTMDAWGQGTSVTVSS

SEQ ID NO: 17 - protein, 107 aa, Artificial Sequence
  VL domain sequence
DIQMTQSPSSLSASLGDRVTITCRSSQDIGNYLTWFQQKVGRSPRRMIYGAIKLEDGVPSRFSGSRSGSDYSLTISSLESEDVADYQCLQSIQYPFTFGSGTKLEIK

SEQ ID NO: 18 - protein, 4 aa, Artificial Sequence
  ITAM (immunoreceptor tyrosine-based activation motif)
  misc_feature (2)..(3): Xaa can be any naturally occurring amino acid
  MISC_FEATURE (4)..(4): Xaa may be Leu or Ile
YXXX

SEQ ID NO: 19 - protein, 120 aa, Artificial Sequence [sequence omitted]

SEQ ID NO: 20 - protein, 111 aa, Artificial Sequence [sequence omitted]

SEQ ID NO: 21 - protein, 113 aa, Artificial Sequence [sequence omitted]

SEQ ID NO: 22 - protein, 113 aa, Artificial Sequence
  VL sequence of anti-PD-1 checkpoint inhibitor Nivolumab
STGEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTV

SEQ ID NO: 23 - protein, 117 aa, Artificial Sequence
  VH sequence of anti-PD-1 checkpoint inhibitor Pidilizumab
QVQLVQSGSELKKPGASVKISCKASGYTFSNYGMNWVRQAPGQGLQWMGWINTDSGESTYAEEFKGRFVFSLDTSVSTAYLQITSLTAEDTGMYFCAKVGYDALDYWGQGTLVTVSS

SEQ ID NO: 24 - protein, 106 aa, Artificial Sequence
  VL sequence of anti-PD-1 checkpoint inhibitor Pidilizumab
EIVLTQSPSSLSASVGDRVTITCSARSSVSYMHWYQQKPGKAPKLWIYRTSNLASGVPSRFSGSGSGTDFTLTINSLQPEDFATYYCQQRSSFPLTFGGGTKLEIK

SEQ ID NO: 25 - protein, 118 aa, Artificial Sequence [sequence omitted]

SEQ ID NO: 26 - protein, 110 aa, Artificial Sequence
  VL sequence of anti-PD-L1 checkpoint inhibitor Atezolizumab
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTV

SEQ ID NO: 27 - protein, 121 aa, Artificial Sequence
  VH sequence of anti-PD-L1 checkpoint inhibitor Durvalumab
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 28 - protein, 111 aa, Artificial Sequence
  VL sequence of anti-PD-L1 checkpoint inhibitor Durvalumab
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTV

SEQ ID NO: 29 - protein, 120 aa, Artificial Sequence
  VH sequence of anti-PD-L1 checkpoint inhibitor Avelumab
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 30 - protein, 110 aa, Artificial Sequence
  VL sequence of anti-PD-L1 checkpoint inhibitor Avelumab
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 31 - protein, 6 aa, Artificial Sequence
  checkpoint inhibitor VH domain CDR
TNYYMY

SEQ ID NO: 32 - protein, 17 aa, Artificial Sequence
  checkpoint inhibitor VH domain CDR
GINPSNGGTNFNEKFKN

SEQ ID NO: 33 - protein, 11 aa, Artificial Sequence
  checkpoint inhibitor VH domain CDR
RDYRFDMGFDY

SEQ ID NO: 34 - protein, 15 aa, Artificial Sequence
  checkpoint inhibitor VL domain CDR
RASKGVSTSGYSYLH

SEQ ID NO: 35 - protein, 7 aa, Artificial Sequence
  checkpoint inhibitor VL domain CDR
LASYLES

SEQ ID NO: 36 - protein, 9 aa, Artificial Sequence
  checkpoint inhibitor VL domain CDR
QHSRDLPLT

(19) United States
(12) Patent Application Publication
(10) Pub. No.: US 2022/0257757 A1
(43) Pub. Date: Aug. 18, 2022

(54) METHOD FOR PRECONDITIONING A SUBJECT WHO IS ABOUT TO RECEIVE A T-CELL THERAPY

(71) Applicant: AUTOLUS LIMITED, London (GB)

(72) Inventors: Nushmia Khokhar, London (GB); Vijay Peddareddigari, London (GB)

(21) Appl. No.: 17/627,266

(22) PCT Filed: Jul. 15, 2020
(86) PCT No.: PCT/GB2020/051704

§ 371 Date: Jan. 14, 2022

(30) Foreign Application Priority Data
Jul. 16, 2019 (GB) 1910185.6
Dec. 5, 2019 (GB) 1917806.0

(51) Int. Cl. A61K 39/395; A61K 31/675; A61K 31/7076; A61K 38/17; A61K 35/17; A61P 35/00

(52) U.S. Cl. CPC A61K 39/3955; A61K 31/675; A61K 31/7076; A61K 2039/505; A61K 35/17; A61P 35/00; A61K 38/1774

(57) ABSTRACT

The present invention provides a method for preconditioning a subject who is about to receive a therapeutic T-cell composition, which comprises the step of administering one or more doses of a checkpoint inhibitor to the subject prior to administration of the therapeutic T-cell composition, wherein the subject does not receive any further doses of the checkpoint inhibitor after administration of the therapeutic T-cell composition.

### FIELD OF THE INVENTION

The present invention relates in general to adoptive cell therapy (ACT) using T cells. In particular, the invention relates to a method for preconditioning a subject who is about to receive a T-cell therapy.

### BACKGROUND TO THE INVENTION

Adoptive cell therapy (ACT) involves administrating disease-relevant immune cells to a subject. For example, where the subject has a cancer, ACT may involve administering immune cells with direct anticancer activity.

ACT using naturally occurring tumour-reactive lymphocytes has mediated durable, complete regressions in patients with melanoma and has also been used in the treatment of epithelial cancers. In addition, the ability to genetically engineer lymphocytes to express conventional T cell receptors (TCRs) or chimeric antigen receptors (CARs) has further extended the successful application of ACT for cancer treatment.

ACT has multiple advantages compared with other forms of cancer immunotherapy which rely on the active in vivo development of sufficient numbers of anti-tumour cells with the function necessary to mediate cancer regression. For use in ACT, large numbers of antitumor lymphocytes (up to 10¹¹) can be readily grown in vitro and selected for high-avidity recognition of the tumour, as well as for the effector functions required to mediate cancer regression. In vitro activation allows such cells to be released from the inhibitory factors that exist in vivo. Also, ACT enables the manipulation of the host before cell transfer to provide a favourable microenvironment that better supports antitumor immunity.

In this respect, it has been shown that preconditioning a patient with one or more immunosuppressive chemotherapy drugs prior to T cell infusion can increase the effectiveness of the transplanted T cells. For example, patients may receive cyclophosphamide and fludarabine as preconditioning to decrease immunosuppressive cells prior to T cell infusion. Pre-conditioning patients prior to T cell therapies with cyclophosphamide and fludarabine improves the efficacy of the T cell therapy by reducing the number of endogenous lymphocytes and increasing the serum level of homeostatic cytokines and/or pro-immune factors present in the patient.

### Immunosuppression

Despite encouraging results in preclinical models and in patients, the existence of a number of different immune-suppressive pathways can restrict the full potential of adoptive T-cell therapy. This includes increased expression of inhibitory immune receptors such as TIM-3, CTLA4 and PD-1 of T cells following T-cell activation, which can limit the duration and strength of the adaptive immune response.

Tumours can evade the immune system by upregulating immunoinhibitory molecules. These so-called immune checkpoints normally serve as a brake on immune cell overactivity and prevent autoimmune reactivity. Tumour acquisition of these properties leads to tumour cell evasion and progression.

The programmed cell death-1 receptor (PD-1) axis has been recognised as a pivotal immune checkpoint. In a tumour, the interaction of PD-1 on tumour infiltrating T cells with its ligands PD-L1 and/or PD-L2 on malignant cells inhibits TIL potency. Immune checkpoint blockade, for example anti-PD1, anti-PD-L1 and anti-CTLA4 has been successfully used in the treatment of various solid tumours to prevent checkpoint molecule triggered exhaustion.

Like their endogenous counterparts, CAR-T cells can also acquire a differentiated and exhausted phenotype associated with increased expression of PD-1. For this reason, various clinical studies are underway in which the patients receive PD-1 or PD-L1 blockade following CAR-T cell infusion.

For example, Chong et al (2017; Blood 129:1039-1041) report a case in which the PD-1 blocking antibody, pembrolizumab, was administered to a patient with refractory diffuse large B-cell lymphoma (DLBCL) 26 days after therapy with CAR-T cells directed against CD19. Pembrolizumab was chosen for therapy because the patient's tumour cells strongly expressed PD-L1. By day 45, significant clinical improvement was noted.

Maude et al (J. Clin. Oncol. 2017, 35, 103) observed that CD19-targeted CAR T cell therapy show complete response (CR) rates of 70-95% in B-cell acute lymphoblastic leukemia (B-ALL), but a subset of patients do not respond or relapse due to poor T cell expansion and persistence. They describe the treatment of four children with relapsed B-ALL with anti-CD19 CAR-T cells followed by 1 to 3 doses of pembrolizumab starting 14 days to 2 months post CAR-T cell infusion. It was found that pembrolizumab increased or prolonged detection of circulating CAR T cells in all four children.

Locke et al (J. Clin. Oncol. 2017, 35, TPS7572) is a study design for a phase 1-2 clinical trial for patients with refractory DLBCL. In view of the expression of PD-L1 on DLBCL cells, the authors hypothesise that PD-1 pathway blockade may result in improved clinical outcomes. The study involved giving the patients a single infusion of anti-CD19 CAR-T cells followed by the anti-PD-L1 antibody atezolizumab every 21 days for four doses.

### DESCRIPTION OF THE FIGURES

FIG. 1—Schematic diagram showing a classical chimeric antigen receptors (a) Basic schema of a chimeric antigen receptor; (b) First generation receptors; (c) Second generation receptors; (d) Third generation receptors.

FIG. 2—Activated T-cells expressing a CD19/CD22 OR gate have upregulated expression of both PD1 and PD-L1.

FIG. 3—Table showing the VH, VL and CDR sequences of various anti-PD1 or anti-PD-L1 checkpoint inhibitors.

FIG. 4—Schematic diagram showing the study design for a Phase 1/2 study of CAR-T cells expressing a CD19/CD22 OR gate in patients with relapsed/refractory Diffuse Large B Cell Lymphoma (r/r DLBCL).

FIG. 5—Swim plot showing preliminary efficacy of Phase 1/2 study of CAR-T cells expressing a CD19/CD22 OR gate in patients with r/r DLBCL.

### SUMMARY OF ASPECTS OF THE INVENTION

The current rationale for giving patients checkpoint blockade after T-cell therapy is to prevent immunosuppression caused by the T cells encountering immunoinhibitory molecules on malignant cells and to reactivate exhausted CAR-T cells once they have encountered antigen.

The present inventors have found that the effect of immune checkpoint blockade when used in combination with an adoptive T cell therapy is equivalent and even improved if the checkpoint inhibitor is given to the subject prior to administration of the T cell therapy. Without wishing to be bound by theory, the present inventors believe this is because the T-cells themselves exert an immunosuppressive effect on each other even before encountering a tumour cell. The presence of an immune checkpoint blockade in the patient prior to administration of the T-cell therapy means that the checkpoint blockade is present from the moment the T-cell therapy is administered. The intra-T cell immunosuppressive effect is therefore alleviated as soon as the T cells are administered to the patient.

Thus, in a first aspect the present inventors provides a method for preconditioning a subject who is about to receive a therapeutic chimeric antigen receptor (CAR) T-cell composition, which comprises the step of administering one or more doses of a checkpoint inhibitor to the subject prior to administration of the CAR therapeutic T-cell composition, wherein the subject does not receive any further doses of the checkpoint inhibitor after administration of the therapeutic CAR T-cell composition.

The checkpoint inhibitor may inhibit the interaction between PD-1 and PD-L1. For example, the checkpoint inhibitor may be an antibody which binds programmed cell death protein 1 (PD-1), such as pembrolizumab.

The checkpoint inhibitor may be administered before, after or together with one or more other pre-conditioning agent(s) such as cyclophosphamide and/or fludarabine.

The checkpoint inhibitor may be administered to the subject in single or multiple doses.

The checkpoint inhibitor may be administered to the subject in a single dose of between 100 and 800 mg, for example about 200 mg.

In a second aspect, the present invention provides a method for treating cancer in a subject which comprises the following steps:

(i) administering one or more doses of a checkpoint inhibitor to the subject; prior to (ii) administering a therapeutic CAR T-cell composition to the subject

wherein the subject does not receive any further doses of the checkpoint inhibitor after administration of the therapeutic CAR T-cell composition.

In the method of the second aspect of the invention, step (i) may be carried out up to three weeks before step (ii). For example, step (i) may be carried out about 1 day before step (ii).

The cancer may be a B cell malignancy such as diffuse large B-cell lymphoma (DLBCL).

In a third aspect, the present invention provides a kit for preconditioning a subject who is about to receive a CAR T-cell therapy, which comprises:
(a) a checkpoint inhibitor
(b) one or more other pre-conditioning agent(s)
   for separate, sequential, simultaneous or combined administration to a subject.

The one or more other preconditioning agents may be cyclophosphamide and/or fludarabine.

The kit may also comprise (c) a therapeutic CAR T-cell composition, and (a) and (b) may be for separate, sequential, simultaneous or combined administration to a subject prior to (c).

In a fourth aspect the present invention provides a checkpoint inhibitor for use in preconditioning a subject who is about to receive a therapeutic CAR T-cell composition, which preconditioning method comprises the step of administering one or more doses of the checkpoint inhibitor to the subject prior to administration of the therapeutic CAR T-cell composition, wherein the subject does not receive any further doses of the checkpoint inhibitor after administration of the therapeutic CAR T-cell composition.

In a fifth aspect the present invention provides a checkpoint inhibitor for use in a method for treating cancer in a subject which method comprises the following steps:

(i) administering one or more doses of the checkpoint inhibitor to the subject; prior to (ii) administering a therapeutic CAR T-cell composition to the subject wherein the subject does not receive any further doses of the checkpoint inhibitor after administration of the therapeutic CAR T-cell composition.

In a sixth aspect the present invention provides the use of a checkpoint inhibitor in the manufacture of a medicament for preconditioning a subject who is about to receive a therapeutic CAR T-cell composition, which preconditioning method comprises the step of administering one or more doses of the checkpoint inhibitor to the subject prior to administration of the therapeutic CAR T-cell composition, wherein the subject does not receive any further doses of the checkpoint inhibitor after administration of the therapeutic CAR T-cell composition.

In a seventh aspect, the present invention provides the use of a checkpoint inhibitor in the manufacture of a medicament for treating cancer in a subject, which method comprises the following steps:

(i) administering one or more doses of the checkpoint inhibitor to the subject; prior to

(ii) administering a therapeutic CAR T-cell composition to the subject

wherein the subject does not receive any further doses of the checkpoint inhibitor after administration of the therapeutic CAR T-cell composition.

### FURTHER ASPECTS OF THE INVENTION

The present invention also relates to the aspects listed in the following numbered paragraphs:

1. A method for preconditioning a subject who is about to receive a therapeutic T-cell composition, which comprises the step of administering one or more doses of a checkpoint inhibitor to the subject prior to administration of the therapeutic T-cell composition, wherein the subject does not receive any further doses of the checkpoint inhibitor after administration of the therapeutic T-cell composition.

2. A method according to paragraph 1, wherein the therapeutic T cell composition comprises tumour infiltrating lymphocytes (TILs) or engineered TCR-expressing T cells.

The following detailed description, as it relates to methods, kits and uses, applies equally to the aspects laid out in the above paragraphs as to the aspects of the invention in the claims.

### DETAILED DESCRIPTION

### Immunotherapy

The present invention relates to a method for preconditioning a subject who is about to receive a cell therapy, such as a T- or NK-cell therapy.

T cell therapies include adoptive T cell therapy, tumour-infiltrating lymphocyte (TIL) immunotherapy, autologous cell therapy, engineered autologous cell therapy, and allogeneic T cell transplantation.

Adoptive T cell therapy includes any method which involves administering T cells to a patient, such that the T-cells survive in the patient and exert their therapeutic function. TIL immunotherapy is a type of adoptive T cell therapy, wherein lymphocytes capable of infiltrating tumour tissue are isolated, enriched in vitro, and administered to a patient. The TIL cells can be either autologous or allogeneic. Autologous cell therapy is an adoptive T cell therapy that involves isolating T cells capable of targeting tumour cells from a patient, enriching the T cells in vitro, and administering the T cells back to the same patient. Allogeneic T cell transplantation can include transplant of naturally occurring T cells expanded ex vivo or genetically engineered T cells. Engineered autologous cell therapy, is an adoptive T cell therapy wherein a patient's own lymphocytes are isolated, genetically modified to express a tumour targeting molecule, expanded in vitro, and administered back to the patient. Non-T cell transplantation can include autologous or allogeneic therapies with non-T cells such as, but not limited to, natural killer (NK) cells.

The cells for use in immunotherapy can come from any source known in the art. For example, T cells can be differentiated in vitro from a hematopoietic stem cell population, or obtained directly from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. Alternatively, the T cells can be derived from one of the available T cell lines.

Engineered autologous cell therapy is a process by which a patient's own T cells are collected and subsequently genetically altered to recognize and target one or more antigens expressed on the cell surface of one or more specific tumor cells or malignancies. T cells can be engineered to express, for example, chimeric antigen receptors (CAR) or non-endogenous T cell receptor (TCR).

### Chimeric Antigen Receptors (CARS)

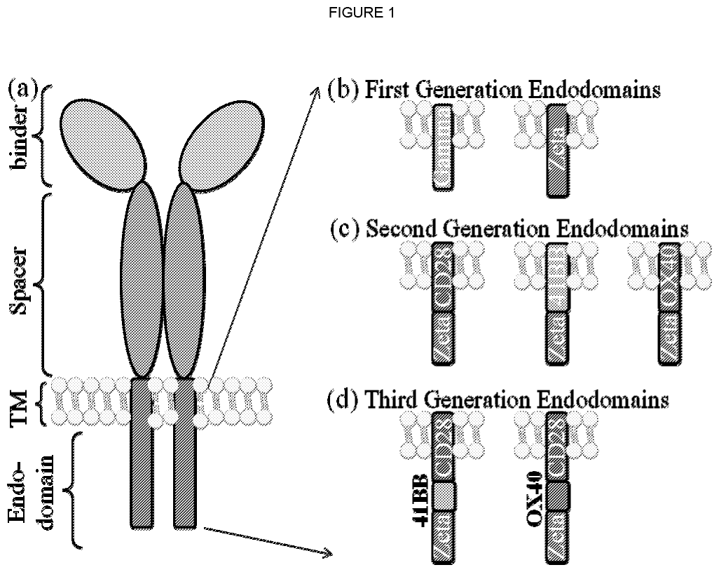

CARs, which are shown schematically in FIG. 1, are chimeric type I transmembrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral or lentiviral vectors to generate cancer-specific T cells for adoptive cell transfer. When the CAR binds the target-antigen, this results in the transmission of an activating signal to the T-cell it is expressed on. Thus, the CAR directs the specificity and cytotoxicity of the T cell towards tumour cells expressing the targeted antigen.

### Tandem CARs (TanCARs)

Bispecific CARs, known as tandem CARs or TanCARs, have been developed to target two or more cancer specific markers simultaneously. In a TanCAR, the extracellular domain comprises two antigen binding specificities in tandem, joined by a linker. The two binding specificities (scFvs) are thus both linked to a single transmembrane portion: one scFv being juxtaposed to the membrane and the other being in a distal position. When a TanCAR binds either or both of the target antigens, this results in the transmission of an activating signal to the cell it is expressed on.

### Antigen Binding Domain

The antigen binding domain is the portion of CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

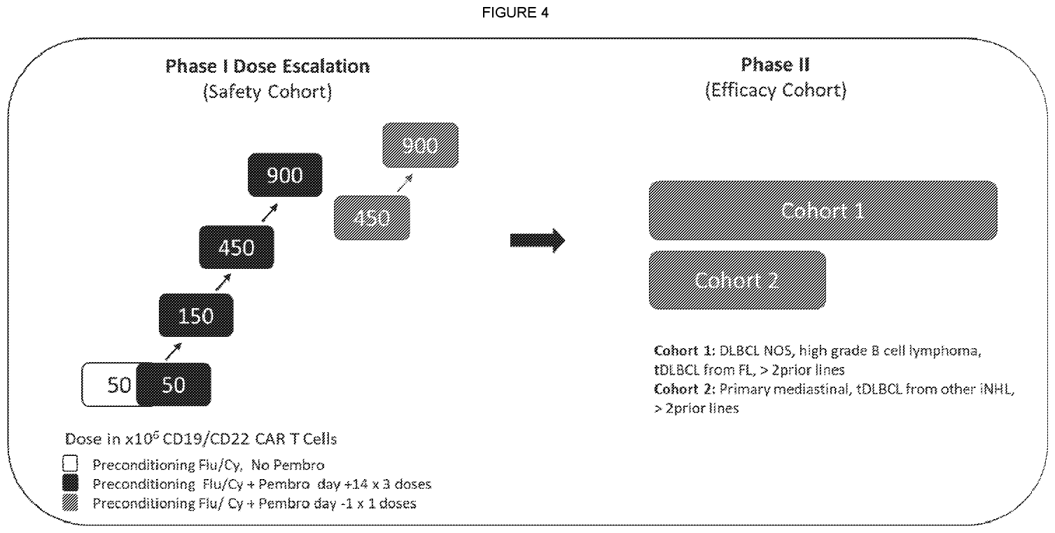

In a classical CAR, the antigen-binding domain comprises: a single-chain variable fragment (scFv) derived from a monoclonal antibody (see FIG. 4c). CARs have also been produced with domain antibody (dAb) or VHH antigen binding domains (see FIG. 4b) or which comprise a Fab fragment of, for example, a monoclonal antibody (see FIG. 4a). A FabCAR comprises two chains: one having an antibody-like light chain variable region (VL) and constant region (CL); and one having a heavy chain variable region (VH) and constant region (CH). One chain also comprises a transmembrane domain and an intracellular signalling domain. Association between the CL and CH causes assembly of the receptor.

The two chains of a Fab CAR may have the general structure:

VH-CH—spacer-transmembrane domain—intracellular signalling domain; and VL-CL
or
VL-CL—spacer-transmembrane domain—intracellular signalling domain; and VH-CH

For Fab-type chimeric receptors, the antigen binding domain is made up of a VH from one polypeptide chain and a VL from another polypeptide chain.

The polypeptide chains may comprise a linker between the VH/VL domain and the CH/CL domains. The linker may be flexible and serve to spatially separate the VH/VL domain from the CH/CL domain.

The antigen-binding domain of the CAR may bind a tumour associated antigen. Various tumour associated antigens (TAA) are known, for example as shown in the following Table 1.

TABLE 1

| Cancer type | TAA |
|---|---|
| Diffuse Large B-cell Lymphoma | CD19, CD20, 0D22 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The or each CAR may bind one of the following target antigens: CD19, CD22, BCMA, PSMA, GD2, CD79 or FCRL5.

### CD19

An antigen binding domain of a CAR which binds to CD19 may comprise a sequence derived from one of the CD19 binders shown in Table 2.

TABLE 2

| Binder | References |
|---|---|
| HD63 | Pezzutto (Pezzutto, A. et al. J. Immunol. Baltim. Md |
| | 1950 138, 2793-2799 (1987) |
| 4g7 | Meeker et al (Meeker, T. C. et al. Hybridoma 3, |
| | 305-320 (1984) |
| Fmc63 | Nicholson et al (Nicholson, I. C. et al. Mol. Immunol. |
| | 34, 1157-1165 (1997) |
| B43 | Bejcek et al (Bejcek, B. E. et al. Cancer Res. 55, |
| | 2346-2351 (1995) |
| SJ25C1 | Bejcek et al (1995, as above) |
| BLY3 | Bejcek et al (1995, as above) |
| B4, or re-surfaced, | Roguska et al (Roguska, M. A. et al. Protein Eng. 9, |
| or humanized B4 | 895-904 (1996) |
| HB12b, optimized | Kansas et al (Kansas, G. S. & Tedder, T. F. J. |
| and humanized | Immunol. Baltim. Md 1950 147, 4094-4102 (1991); |
| | Yazawa et al (Yazawa et al Proc. Natl. Acad. Sci. |
| | U.S.A. 102, 15178-15183 (2005); Herbst et al |
| | (Herbst, R. et al. J. Pharmacol. Exp. Ther. 335, |
| | 213-222 (2010) |

Alternatively a CAR which binds CD19 may have an antigen-binding domain which comprises:
a) a heavy chain variable region (VH) having complementarity determining regions (CDRs) with the following sequences:

CDR1-
(SEQ ID No. 1)
GYAFSSS;
CDR2-
(SEQ ID No. 2)
YPGDED
CDR3-
(SEQ ID No. 3)
SLLYGDYLDY;

and
b) a light chain variable region (VL) having CDRs with the following sequences:

CDR1-
(SEQ ID No. 4)
SASSSVSYMH;
CDR2-
(SEQ ID No. 5)
DTSKLAS
CDR3-
(SEQ ID No. 6)
QQWNINPLT.

The antigen binding domain may comprise a VH domain having the sequence shown as SEQ ID No. 7 and a VL domain having the sequence shown as SEQ ID No 8.

-VH sequence
SEQ ID No. 7
QVQLQQSGPELVKPGASVKISCKASGYAFSSSWMNWVKQRPGKGLEWIG
RIYPGDEDTNYSGKFKDKATLTADKSSTTAYMQLSSLTSEDSAVYFCAR
SLLYGDYLDYWGQGTTLTVSS
-VL sequence
SEQ ID No 8
QIVLTQSPAIMSASPGEKVTMTCSASSSVSYMHWYQQKSGTSPKRWIYD
TSKLASGVPDRFSGSGSGTSYFLTINNMEAEDAATYYCQQWNINPLTFG
AGTKLELKR

### CD22

A CAR which binds to CD22 may have an antigen domain derived from m971, HA22 or BL22 as described by Haso et al. (Blood; 2013; 121(7)).

Alternatively, a CAR which binds CD22 may have an antigen binding domain as described in United Kingdom application No. 1809773.3, such as one which comprises:

a) a heavy chain variable region (VH) having complementarity determining regions (CDRs) with the following sequences:

CDR1-
(SEQ ID No. 10)
NFAMA
CDR2-
(SEQ ID No. 11)
SISTGGGNTYYRDSVKG
CDR3-
(SEQ ID No. 12)
QRNYYDGSYDYEGYTMDA;

and

b) a light chain variable region (VL) having complementarity determining regions (CDRs) with the following sequences:

CDR1-
(SEQ ID No. 13)
RSSQDIGNYLT
CDR2-
(SEQ ID No. 14)
GAIKLED
CDR3-
(SEQ ID No. 15)
LQSIQYP

The antigen binding domain of a CD22 CAR may comprise a VH domain having the sequence shown as SEQ ID No. 16; and a VL domain having the sequence shown as SEQ ID No. 17.

SEQ ID No. 16
EVQLVESGGGLVQPGRSLKLSCAASGFTFSNFAMAWVRQPPTKGLEWVA
SISTGGGNTYYRDSVKGRFTISRDDAKNTQYLQMDSLRSEDTATYYCAR
QRNYYDGSYDYEGYTMDAWGQGTSVTVSS
SEQ ID No. 17
DIQMTQSPSSLSASLGDRVTITCRSSQDIGNYLTWFQQKVGRSPRRMIY
GAIKLEDGVPSRFSGSRSGSDYSLTISSLESEDVADYQCLQSIQYPFTF
GSGTKLEIK

Intracellular T Cell Signaling Domain (Endodomain)

The CAR may comprise or associate with an activating endodomain: the signal-transmission portion of the CAR. After antigen recognition, receptors cluster and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, the endodomains from CD28, 4-1BB or OX40 can be used with CD3-Zeta to transmit a proliferative/survival signal, or three can be used together, e.g. OX-40/CD28/CD3z or 4-1BB/CD28/CD3z. A costimulatory signaling region may be or comprise the signaling region of CD28, OX-40, 4 IBB, CD27, inducible T cell costimulator (ICOS), CD3 gamma, CD3 delta, CD3 epsilon, CD247, Ig alpha (CD79a), or Fc gamma receptor.

The endodomain may comprise:

(i) an ITAM-containing endodomain, such as the endodomain from CD3 zeta; and/or

(ii) a co-stimulatory domain, such as the endodomain from CD28; and/or

(iii) a domain which transmits a survival signal, for example a TNF receptor family endodomain such as OX-40 or 4-1BB.

An endodomain which contains an ITAM motif can act as an activation endodomain in this invention. Several proteins are known to contain endodomains with one or more ITAM motifs. Examples of such proteins include the CD3 epsilon chain, the CD3 gamma chain and the CD3 delta chain to name a few. The ITAM motif can be easily recognized as a tyrosine separated from a leucine or isoleucine by any two other amino acids, giving the signature YxxL/I (SEQ ID NO. 18). Typically, but not always, two of these motifs are separated by between 6 and 8 amino acids in the tail of the molecule (YxxL/Ix(6-8)YxxL/I). Hence, one skilled in the art can readily find existing proteins which contain one or more ITAM to transmit an activation signal. Further, given the motif is simple and a complex secondary structure is not required, one skilled in the art can design polypeptides containing artificial ITAMs to transmit an activation signal (see WO 2000/063372, which relates to synthetic signalling molecules).

A number of systems have been described in which the antigen recognition portion of the CAR is on a separate molecule from the signal transmission portion, such as those described in WO015/150771; WO2016/124930 and WO2016/030691. One or more of the viral vectors used in the method of the invention may encode such a “split CAR”. Alternatively one vector may comprise a nucleic acid sequence encoding the antigen recognition portion and one vector may comprise a nucleic acid sequence encoding the intracellular signalling domain.

Where the composition of viral vectors includes more than one vector comprising a nucleic acid sequence encoding a CAR, the CARs may have different endodomains or different endodomain combinations. For example, one CAR may be a second generation CAR and one CAR may be a third generation CAR. Alternatively, both CARs may be a second generation CAR but may have different co-stimulatory domains. For example, different second generation CAR signalling domains include: 41BB-CD3ζ; OX40-CD3 and CD28-CD3ζ.

### Signal Peptide

One or more nucleic acid sequences in the vector composition may encode a signal peptide so that when the CAR or activity modulator is expressed inside a cell, the nascent protein is directed to the endoplasmic reticulum and subsequently to the cell surface, where it is expressed (or secreted).

The core of the signal peptide may contain a long stretch of hydrophobic amino acids that tends to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

A CAR may have the general formula:

Signal peptide—antigen binding domain—spacer domain—transmembrane domain—intracellular T cell signaling domain (endodomain).

### Spacer

The CAR may comprise a spacer sequence to connect the antigen binding domain with the transmembrane domain and spatially separate the antigen binding domain from the endodomain. A flexible spacer allows to the antigen binding domain to orient in different directions to enable antigen binding.

The spacer sequence may, for example, comprise an IgG1 Fc region, an IgG1 hinge or a CD8 stalk, or a combination thereof. The spacer may alternatively comprise an alternative sequence which has similar length and/or domain spacing properties as an IgG1 Fc region, an IgG1 hinge or a CD8 stalk.

Where the composition of viral vectors includes more than one vector comprising a nucleic acid sequence encoding a CAR, the CARs may have different spacers.

### OR Gates

The T cells used in the method of the present invention may comprise two or more CARs. This may be as a result of transduction with two or more vectors, each comprising a nucleic acid sequence encoding a CAR; or it may be as a result of transduction with a single vector which comprises a nucleic acid construct encoding two or more CARs.

A CAR may be used in a combination with one or more other activatory or inhibitory chimeric antigen receptors. For example, they may be used in combination with one or more other CARs in a “logic-gate”, a CAR combination which, when expressed by a cell, such as a T cell, are capable of detecting a particular pattern of expression of at least two target antigens. If the at least two target antigens are arbitrarily denoted as antigen A and antigen B, the three possible options are as follows:

“OR GATE”—T cell triggers when either antigen A or antigen B is present on the target cell

“AND GATE”—T cell triggers only when both antigens A and B are present on the target cell

“AND NOT GATE”—T cell triggers if antigen A is present alone on the target cell, but not if both antigens A and B are present on the target cell

Engineered T cells expressing these CAR combinations can be tailored to be exquisitely specific for cancer cells, based on their particular expression (or lack of expression) of two or more markers.

Such “Logic Gates” are described, for example, in WO2015/075469, WO2015/075470 and WO2015/075470.

An “OR Gate” comprises two or more activatory CARs each directed to a distinct target antigen expressed by a target cell. The advantage of an OR gate is that the effective targetable antigen is increased on the target cell, as it is effectively antigen A+antigen B. This is especially important for antigens expressed at variable or low density on the target cell, as the level of a single antigen may be below the threshold needed for effective targeting by a CAR-T cell. Also, it avoids the phenomenon of antigen escape. For example, some lymphomas and leukemias become CD19 negative after CD19 targeting: using an OR gate which targets CD19 in combination with another antigen provides a “back-up” antigen, should this occur. The “back up” antigen may be CD22, as described in WO2016/102965.

The T cells used in the method of the invention may express an “OR gate” comprising an anti-CD19 CAR and an anti-CD22 CAR. The two CARs may have different endodomains, for example one CAR may have a 4-1BB/CD3z second generation endodomain and the other CAR may have a CD28/CD3z second generation endodomain. Alternatively the two CARs may have the same second or third generation endodomains.

### Transgenic T-Cell Receptor

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using vectors. For example the genes for engineered TCRs may be reintroduced into autologous T cells and transferred back into patients for T cell adoptive therapies. Such ‘heterologous’ TCRs may also be referred to herein as ‘transgenic TCRs’.

### Checkpoint Inhibitors

In a natural immune response, anti-tumour T cell responses occur upon binding of T cell receptors (TCR) to tumour-specific antigens, causing them to proliferate, differentiate and eventually eradicate cells expressing these antigens. This TCR-mediated activity is regulated by both co-stimulatory and co-inhibitory molecules. Otherwise known as immune checkpoints, these negative regulators of activation and maintenance functions in T-cells usually serve to prevent autoimmunity and maintain immune homeostasis.

Following immune activation, various inhibitory checkpoint molecules such as cytotoxic T-lymphocyte associated protein 4 (CTLA-4), and programmed cell death 1 (PD-1) are expressed by T-cells. Binding of these molecules to their corresponding ligands activates suppressive immune checkpoint pathways, leading to the attenuation and termination of T cell activity. These inhibitory checkpoint ligands are often overexpressed by tumour cells and antigen presenting cells (APCs) in the tumour microenvironment, and thus play a role in facilitating immune attack evasion and tumour progression.

Checkpoint inhibitors are molecules which block the interaction between inhibitory checkpoint molecules with their ligands. In the context of cancer, the use of checkpoint inhibitors has been described as a strategy to increase T-cell responses in the tumour microenvironment, with a view to enabling the subject's immune system to more effectively recognise and eradicate tumours. As checkpoint inhibitors function by targeting the patient's own immune system rather than tumour cells themselves, they have the potential to be effective for a wide range of malignancies and are not necessarily specific to any particular type of cancer.

Ipilimumab, an anti-CTLA-4 antibody, was the first checkpoint inhibitor to gain approval by the US Food and Drug Administration (FDA) in 2011, for the treatment of melanoma. Since then, there has been a surge in the clinical development of various checkpoint inhibitors targeting both co-inhibitory and co-stimulatory checkpoints such as PD-1, PD-L1, CD520 and CD20, for an expanding list of indications.

The PD-1 receptor has been identified as a dominant inhibitory immune checkpoint, and is expressed on activated T cells, B cells and myeloid cells. Upon engagement with its corresponding ligand PD-L1, present on the surface of APCs and tumour cells, various immunosuppressive responses are induced. These include impairment of inflammatory cytokine production, cell cycle arrest, diminished transcription of cell survival proteins such as Bcl-XL, desphosphorylation of ZAP70, and phosphorylation of PI3K by recruitment of SHP1 and SHP2 phosphates.

PD-L1 is a molecule which is frequently upregulated in tumour cells in response to the presence of local inflammatory cytokines such as interferon gamma (IFNγ) produced by tumour infiltrating inflammatory cells. The acquisition of this property in the tumour microenvironment therefore acts as an immunosuppressant, preventing effective immune attack.

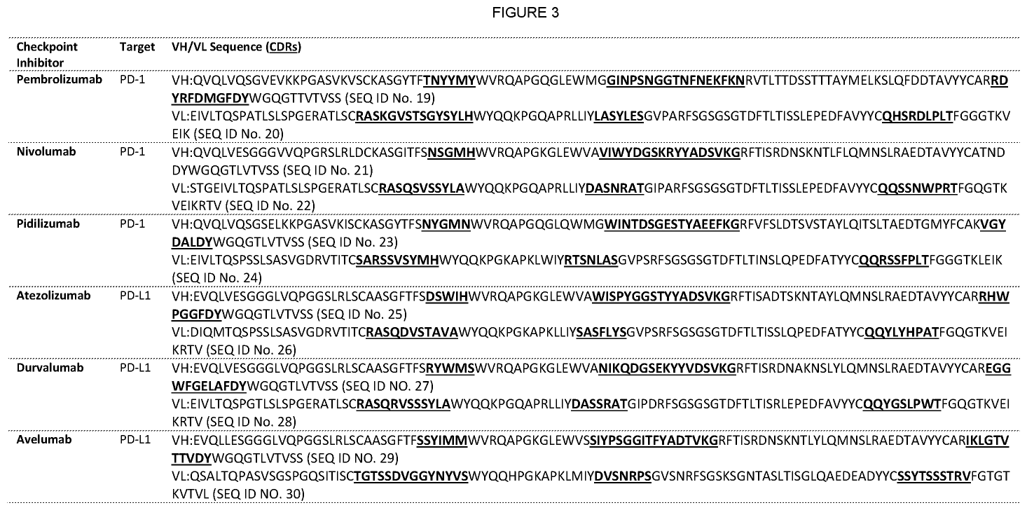

Various antibodies which inhibit this checkpoint by blocking either PD-1 or PD-L1 have been described, some of which are summarised in FIG. 3. Among these inhibitors, Pembrolizumab has been the most widely investigated. In 2014, it was the first anti-PD-1 inhibitor to gain approval from the FDA for the treatment of melanoma and has since been approved for single or combined therapy regimes for indications such as non-small-cell lung carcinoma (NSCLC), head and neck squamous cell carcinoma (HNSCC), renal cell carcinoma (RCC) and cervical cancer, among others. Additional checkpoint inhibitors include Nivolumab and Pidilizumab which target PD-1, and Atezolizumab, Durvalumab and Avelumab which target PD-L1.

The method of the present invention involved administration of a checkpoint inhibitor to a subject. The checkpoint inhibitor may bind to one of the following molecules or its ligand: A2AR (Adenosine A2A receptor); B7-H3: B7-H4; BTLA (B and T Lymphocyte Attenuator); CTLA-4 (Cytotoxic T-Lymphocyte-Associated protein 4); IDO (Indoleamine 2,3-dioxygenase) TDO (tryptophan 2,3-dioxygenase); KIR (Killer-cell Immunoglobulin-like Receptor); LAG3 (Lymphocyte Activation Gene-3); NOX2 (nicotinamide adenine dinucleotide phosphate NADPH oxidase isoform 2); PD-1 (Programmed Death 1 (PD-1) receptor or one of its ligands, PD-L1 and PD-L2; TIM-3 (T-cell Immunoglobulin domain and Mucin domain 3); VISTA (V-domain Ig suppressor of T cell activation); SIGLEC7 (Sialic acid-binding immunoglobulin-type lectin 7); and SIGLEC9 (Sialic acid-binding immunoglobulin-type lectin 9).

The checkpoint inhibitor may bind PD-1, PD-L1 or PD-L2. The checkpoint inhibitor may bind PD-1.

The checkpoint inhibitor may comprise a VH domain with the following complementarity determining regions (CDRs):

(SEQ ID No. 31)
TNYYMY;
(SEQ ID No. 32)
GINPSNGGTNFNEKFKN;
(SEQ ID No. 33)
RDYRFDMGFDY

The checkpoint inhibitor may comprise a VL domain with the following CDRs:

(SEQ ID No. 34)
RASKGVSTSGYSYLH
(SEQ ID No. 35)
LASYLES
(SEQ ID No. 36)
QHSRDLPLT

The checkpoint inhibitor may comprise a VH domain having the sequence shown in FIG. 3 as SEQ ID No. 19 and/or a VH domain having the sequence shown in FIG. 3 as SEQ ID No. 20.

### Preconditioning

The term “pre-conditioning” means preparing a patient who is about to receive a T cell therapy. In the method of the present invention, a checkpoint inhibitor is administered as, or as part of, the preconditioning regimen.

The preconditioning may enhance the effector function of T-cells administered after the checkpoint inhibitor. The preconditioning may reduce or reverse inhibition of T-cell function by immunoinhibitory receptors such as PD-L1.

The preconditioning regimen may involve administration of additional pre-conditioning agents such as cyclophosphamide and/or fludarabine.

Cyclophosphamide (E DOXAN®, CYTOXAN®, PROCYTOX®, NEOSAR®, REVIMMUNE®, CYCLOBLASTIN®) is a nitrogen mustard-derivative alkylating agent with potent immunosuppressive activity. Cyclophosphamide acts as an antineoplastic, and it is used to treat various types of cancers including lymphoma, multiple myeloma, leukemia, mycosis fungoides, neuroblastoma, ovarian cancer, eye cancer, and breast cancer, as well as autoimmune disorders.

Once administered to a patient, cyclophosphamide is converted into acrolein and phosphoramide in the liver. Together, these metabolites crosslink DNA in both resting and dividing cells by adding an alkyl group to guanine bases of DNA at the number seven nitrogen atom of the imidazole ring. As a result, DNA replication is inhibited leading to cell death. In the present invention, the dose of cyclophosphamide can be adjusted depending on the desired effect, e.g., to modulate the reduction of endogenous lymphocytes and/or control the severity of adverse events.

Fludarabine phosphate (FLUDARA®) is a synthetic purine nucleoside that differs from physiologic nucleosides in that the sugar moiety is arabinose instead of ribose or deoxyribose. Fludarabine acts as a purine antagonist antimetabolite, and it is used to treat various types of hematological malignancies, including various lymphomas and leukemias.

Once administered to a patient, fludarabine is rapidly dephosphorylated to 2-fluoro-ara-A and then phosphorylated intracellularly by deoxycytidine kinase to the active triphosphate, 2-fluoro-ara-ATP. This metabolite then interferes with DNA replication, likely by inhibiting DNA polymerase alpha, ribonucleotide reductase, and DNA primase, thus inhibiting DNA synthesis. As a result, fludarabine administration leads to increased cell death in dividing cells.

Preconditioning may have one or more of the following effects: reducing the number of endogenous lymphocytes, removing a cytokine sink, increasing a serum level of one or more homeostatic cytokines or pro-inflammatory factors, enhancing an effector function of T cells administered after the conditioning, enhancing antigen presenting cell activation and/or availability, or any combination thereof prior to a T cell therapy. Preconditioning may involve increasing a serum level of one or more cytokines, e.g., interleukin 7 (IL-7), interleukin 15 (IL-15), interleukin 10 (IL-10), interleukin 5 (IL-5), gamma-induced protein 10 (IP-10), interleukin 8 (IL-8), monocyte chemotactic protein 1 (MCP-1), placental growth factor (PLGF), C-reactive protein (CRP), soluble intercellular adhesion molecule 1 (sICAM-1), soluble vascular adhesion molecule 1 (sVCAM-1), or any combination thereof.

As mentioned above, preconditioning may reduce the number of endogenous lymphocytes. The endogenous lymphocytes that are reduced can include, but are not limited to, endogenous regulatory T cells, B cells, natural killer cells, CD4+ T cells, CD8+ T cells, or any combination thereof, which can inhibit the anti-tumor effect of adoptively transferred T cells. Endogenous lymphocytes can compete with adoptively transferred T cells for access to antigens and supportive cytokines. Preconditioning can remove this competition, resulting in an increase in the level of endogenous cytokines. Once the adoptively transferred T cells are administered to the patient, they are exposed to increased levels of endogenous homeostatic cytokines or pro-inflammatory factors. In addition, cyclophosphamide and fludarabine preconditioning can cause tumor cell death, leading to increased tumor antigen in the patient's serum. This can enhance antigen-presenting cell activation and or availability in the patient, prior to receiving a T cell therapy. Preconditioning can modify the immune environment through induction of molecules that can favour the homeostatic expansion, activation and trafficking of T cells.

### Dosage Regimes

The method of the invention involves administering one or more doses of a checkpoint inhibitor to a subject prior to administration of a therapeutic T-cell composition.

The checkpoint inhibitor may be administered to the subject in single or multiple doses.

Where the checkpoint inhibitor is administered in a single dose, the dose may be 50 to 1000 mg, 100 to 800 mg, 150-600 mg or 200-300 mg or about 200 mg.

Where the checkpoint inhibitor is administered in multiple doses, the patient may receive, for example, 2 to 6; 2 to 4; or about 3 doses. Each dose may be, for example 100 to 300 mg; or about 200 mg. The combined amount of checkpoint inhibitor given over the plurality of doses may be 200 to 1500 mg; 300 to 1200 mg; 500 to 1000 mg; 600 to 800 mg; or about 600 mg. The patient may, for example, receive three doses of 200 mg.

The single or multiple doses of checkpoint inhibitor may be given at any time prior to the T-cell therapy. for example, the checkpoint inhibitor may be given up to one week, up to two weeks or up to three weeks before the T cell therapy. Administration of the checkpoint inhibitor may be or may begin at least seven days, at least six days, at least five days, at least four days, at least three days, at least two days, or at least one day prior to the administration of the T cell therapy. Alternatively administration of the checkpoint inhibitor may be or may begin at least eight days, at least nine days, at least ten days, at least eleven days, at least twelve days, at least thirteen days, or at least fourteen days prior to the administration of the T cell therapy.

The day that a T cell therapy is administered may be designated as day 0. The dose or doses of checkpoint inhibitor may therefore be administered on any of days −1 to −21. The or a dose of checkpoint inhibitor may be given on day 0, provided that it is administered prior to, or at the same time as, the T-cell therapy. In particular, the or a dose of checkpoint inhibitor may be given on day −1.

The patient may also receive one or more doses or one or more additional pre-conditioning agent(s). The additional pre-conditioning agent(s) may be or include cyclophosphamide and/or fludarabine. The additional preconditioning agents may be given together or separately and may be given at any point prior to the T cell therapy. For example, administration of the additional pre-conditioning agent(s) may begin at least seven days, at least six days, at least five days, at least four days, at least three days, at least two days, or at least one day prior to the administration of the T cell therapy. Alternatively, administration of the additional pre-conditioning agent(s) may begin at least eight days, at least nine days, at least ten days, at least eleven days, at least twelve days, at least thirteen days, or at least fourteen days prior to the administration of the T cell therapy.

Cyclophosphamide may be at a dose of about 100, 200, 300, 400, 500, 600 or 700 mg/m². It may be given in single or multiple doses. The total amount of cyclophosphamide given may be 600-1500, 800-1400 or 1000-1200 mg/m². Multiple doses may, for example, be 2, 3, 4 or 5 doses. Spacing between doses may be one or more days. In particular the patient may receive 500 mg/m² cyclophosphamide for two days ending 3 days before administration of the T cell therapy; or 300 mg/m2 cyclophosphamide for three days, ending 3 or 4 days before administration of the T cell therapy.

Fludarabine may be at a dose of about 10, 20, 30, 40, 50 or 60 mg/m². It may be given in single or multiple doses. The total amount of fludarabine given may be 50-150; 60-120 or about 90 or about 120 mg/m². Multiple doses may, for example, be 2, 3, 4, 5 or 6 doses. Spacing between doses may be one or more days. In particular the patient may receive 30 mg/m² fludarabine for two or three days ending 2 to 4 days before administration of the T cell therapy.

The T cell therapy included in the present invention involves the transfer of T cells to a patient. The T cells can be administered at a therapeutically effective amount. For example, a therapeutically effective amount of T cells, e.g., engineered CAR+ T cells or engineered TCR+ T cells, can be at least about 10⁴ cells, at least about 10⁵ cells, at least about 10⁶ cells, at least about 10⁷ cells, at least about 10⁸ cells, at least about 10⁹, or at least about 10¹⁰ cells. In particular, the patient may receive between 10 and 1000 million T cells; or between 50 and 900 million T cells. The patient may receive about 150 million, about 450 million or about 900 million T cells.

### Kits

The present invention also provides kits for use in the methods of the invention. The kit may comprise:
(a) one or more doses of a checkpoint inhibitor
(b) one or more doses of one or more other pre-conditioning agent(s).

The dose(s) of checkpoint inhibitor and other pre-conditioning agent(s) may be for separate, sequential, simultaneous or combined administration to a subject.

Examples of other preconditioning agents which may be present in the kit of the invention are cyclophosphamide and/or fludarabine.

The kit may also comprise one or more doses of a therapeutic T cell composition, such as a T-cell composition expressing a CAR or engineered TCR.

The number of doses and amount in each dose of checkpoint inhibitor/additional pre-conditioning agent(s)/T cell therapy may be suitable for use in the dosage regimes outlined in the previous section.

For example, where the patient is to receive fludarabine for 3 days; cyclophosphamaide for three days ending 3 to 4 days before infusion of CAR-T cells, and one dose of checkpoint inhibitor the day before infusion of CAR-T cells, the kit may comprise:
three doses of fludarabine
three doses of cyclophosphamide, and
one dose of checkpoint inhibitor.

The kit may comprise instructions for use indicating, for example the timing order and route of administration of the one or more doses of a checkpoint inhibitor; the one or more doses of one or more other pre-conditioning agent(s) and optionally the one or more doses of a therapeutic T cell composition.

### Methods of Treatment

The method of the invention may be used to treat cancer. The cancer can be selected from a tumour derived from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, T-cell rich B cell lymphoma (TCRBCL), Primary mediastinal large B cell lymphoma (PMBCL), non-Hodgkin's lymphoma, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukaemia, acute myeloid leukaemia, chronic myeloid leukaemia, acute lymphoblastic leukaemia, chronic lymphocytic leukaemia, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, environmentally induced cancers including those induced by asbestos, and combinations of said cancers.

The method can be used to treat a tumour, wherein the tumour is a lymphoma or a leukaemia. Lymphoma and leukaemia are cancers of the blood that specifically affect lymphocytes. All leukocytes in the blood originate from a single type of multipotent hematopoietic stem cell found in the bone marrow. This stem cell produces both myeloid progenitor cells and lymphoid progenitor cell, which then give rise to the various types of leukocytes found in the body. Leukocytes arising from the myeloid progenitor cells include T lymphocytes (T cells), B lymphocytes (B cells), natural killer cells, and plasma cells. Leukocytes arising from the lymphoid progenitor cells include megakaryocytes, mast cells, basophils, neutrophils, eosinophils, monocytes, and macrophages. Lymphomas and leukaemias can affect one or more of these cell types in a patient.

The method can be used to treat a lymphoma or a leukaemia, wherein the lymphoma or leukaemia is a B cell malignancy. The lymphoma or leukaemia may be selected from B-cell chronic lymphocytic leukaemia/small cell lymphoma, B-cell prolymphocytic leukaemia, lymphoplasmacytic lymphoma (e.g., Waldenstrom macroglobulinemia), splenic marginal zone lymphoma, hairy cell leukaemia, plasma cell neoplasms (e.g., plasma cell myeloma (i.e., multiple myeloma), or plasmacytoma), extranodal marginal zone B cell lymphoma (e.g., MALT lymphoma), nodal marginal zone B cell lymphoma, follicular lymphoma (FL), transformed follicular lymphoma (TFL), primary cutaneous follicle centre lymphoma, mantle cell lymphoma, diffuse large B cell lymphoma (DLBCL), Epstein-Barr virus-positive DLBCL, lymphomatoid granulomatosis, primary mediastinal (thymic) large B-cell lymphoma (PMBCL), Intravascular large B-cell lymphoma, ALK+ large B-cell lymphoma, plasmablastic lymphoma, primary effusion lymphoma, large B-cell lymphoma arising in HHV8-associated multicentric Castleman's disease, Burkitt lymphoma/leukaemia, T-cell prolymphocytic leukaemia, T-cell large granular lymphocyte leukaemia, aggressive NK cell leukaemia, adult T-cell leukaemia/lymphoma, extranodal NK/T-cell lymphoma, enteropathy-associated T-cell lymphoma, Hepatosplenic T-cell lymphoma, blastic NK cell lymphoma, Mycosis fungoides/Sezary syndrome, Primary cutaneous anaplastic large cell lymphoma, Lymphomatoid papulosis, Peripheral T-cell lymphoma, Angioimmunoblastic T cell lymphoma, Anaplastic large cell lymphoma, B-lymphoblastic leukaemia/lymphoma, B-lymphoblastic leukaemia/lymphoma with recurrent genetic abnormalities, T-lymphoblastic leukaemia/lymphoma, and Hodgkin lymphoma. In some embodiments, the cancer is refractory to one or more prior treatments, and/or the cancer has relapsed after one or more prior treatments.

In particular, the cancer may be selected from follicular lymphoma, transformed follicular lymphoma, diffuse large B cell lymphoma, and primary mediastinal (thymic) large B-cell lymphoma. In one particular embodiment, the cancer is diffuse large B cell lymphoma.

The cancer may be refractory to or may have relapsed following one or more of chemotherapy, radiotherapy, immunotherapy (including a T cell therapy and/or treatment with an antibody or antibody-drug conjugate), an autologous stem cell transplant, or any combination thereof. In particular, the cancer may be refractory diffuse large B cell lymphoma.

The invention will now be further described by way of Examples, which are meant to serve to assist one of ordinary skill in the art in carrying out the invention and are not intended in any way to limit the scope of the invention.

### EXAMPLES

### Example 1—Investigating the Expression of PD-L1 by T Cells Expressing a CD19/CD22 OR Gate

T cells were either left untransduced or transduced with a vector co-expressing a CD19 CAR having an antigen-binding domain comprising the VH sequence shown as SEQ ID No. 7 and the VL sequence shown as SEQ ID No. 8; and a CD22 CAR having an antigen-binding domain comprising the VH sequence shown as SEQ ID No. 16 and the VL sequence shown as SEQ ID No. 17.

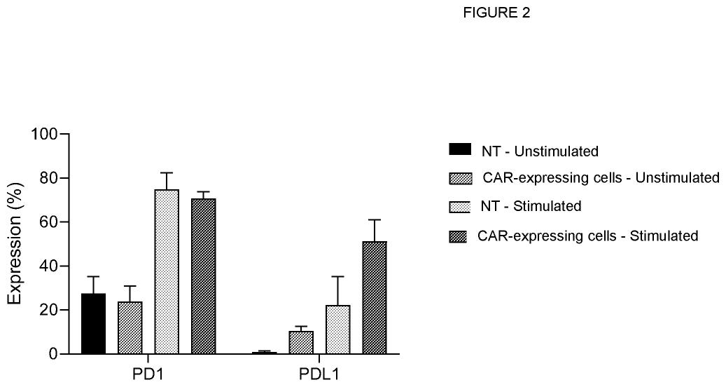

The cells were then activated by stimulation with aCD3 aCD28 beads in the presence of IL2 for 48 hours, following which the expression of PD-1 and PD-L1 by the T-cells was investigated by flow cytometry. The results are shown in FIG. 2. The expression of PD-1 was upregulated on both non-transduced and CAR-expressing T cells following activation. Upregulation of PD-L1 expression was observed for CAR-expressing cells even in the absence of stimulation. For stimulated T cells, PD-L1 upregulation was greater for CAR-expressing cells than untransduced cells.

### Example 2—a Phase 1/2 Study of CAR-T Cells Expressing a CD19/CD22 OR Gate in Patients with Relapsed/Refractory Diffuse Large B Cell Lymphoma (r/r DLBCL) with Two Different Pembrolizumab Regimens

CAR-T cells expressing the CD19/CD22 OR gate described in Example 1 were used in a Phase 1/2 study in patients with relapsed/refractory Diffuse Large B Cell Lymphoma (r/r DLBCL). A dose escalation protocol was followed, as illustrated in FIG. 4, with two different pembrolizumab regimens.

The first three patients, receiving a 50×10⁶ dose of CAR-T cells, did not receive pembrolizumab. The second group of patients received CAR-T cells at one of the following doses: 50×10⁶, 150×10⁶, 450×10⁶ or 900×10⁶ cells, followed by 3×200 mg doses of pembrolizumab: one on day 14, day 35 and day 56. The third group of patients received a single dose of 200 mg pembrolizumab the day before CAR-T cells. They then received CAR-T cells at one of the following doses: 450×10⁶ or 900×10⁶ cells.

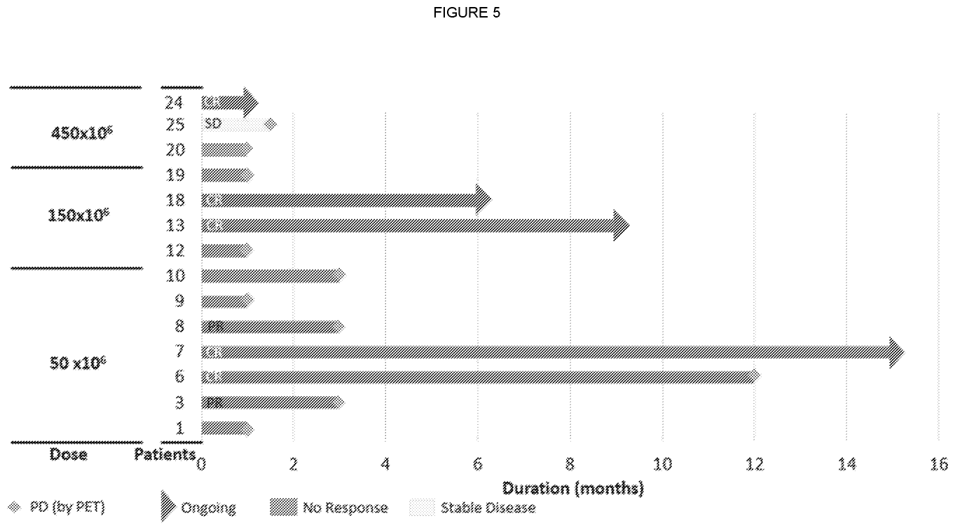

Preliminary results from the first and second groups of patients are shown in FIG. 5. Patients 1, 3 and 6 did not receive pembrolizumab. The remaining patients received three doses of pembrolizumab, starting on day 14 after CAR-T cell infusion.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for preconditioning a subject who is about to receive a therapeutic chimeric antigen receptor (CAR) T-cell composition, which comprises the step of administering one or more doses of a checkpoint inhibitor to the subject prior to administration of the therapeutic CAR T-cell composition, wherein the subject does not receive any further doses of the checkpoint inhibitor after administration of the therapeutic CAR T-cell composition.

2. A method according to claim 1, wherein the checkpoint inhibitor inhibits the interaction between PD-1 and PD-L1.

3. A method according to claim 2, wherein the checkpoint inhibitor is an antibody which binds programmed cell death protein 1 (PD-1).

4. A method according to claim 3, wherein the antibody is pembrolizumab.

5. A method according to any preceding claim, wherein the checkpoint inhibitor is administered before, after or together with one or more other pre-conditioning agent(s).

6. A method according to claim 5, wherein the one or more other preconditioning agents are cyclophosphamide and/or fludarabine.

7. A method according to any preceding claim, wherein the checkpoint inhibitor is administered to the subject in single or multiple doses.

8. A method according to claim 7, wherein the checkpoint inhibitor is administered to the subject in a single dose of between 100 and 800 mg.

9. A method according to claim 8, wherein the single dose of checkpoint inhibitor is about 200 mg.

10. A method for treating cancer in a subject which comprises the following steps:
(i) administering one or more doses of a checkpoint inhibitor to the subject; prior to
(ii) administering a therapeutic CAR Tcell composition to the subject wherein the subject does not receive any further doses of the checkpoint inhibitor after administration of the therapeutic CAR T-cell composition.

11. A method according to claim 10 wherein step (i) is carried out up to three weeks before step (ii).

12. A method according to claim 11, wherein step (i) is carried out about 1 day before step (ii).

13. A method according to any of claims 10 to 12, wherein the cancer is diffuse large B-cell lymphoma (DLBCL).

14. A kit for preconditioning a subject who is about to receive a CAR T-cell therapy, which comprises:
(a) a checkpoint inhibitor
(b) one or more other pre-conditioning agent(s)
for separate, sequential, simultaneous or combined administration to a subject.

15. A kit according to claim 16, wherein the one or more other preconditioning agents are cyclophosphamide and/or fludarabine.

16. A kit according to claim 15 or 16, which also comprises:
(c) a therapeutic CAR T-cell composition
wherein (a) and (b) are for separate, sequential, simultaneous or combined administration to a subject prior to (c).

17. A checkpoint inhibitor for use in preconditioning a subject who is about to receive a therapeutic CAR T-cell composition, which preconditioning method comprises the step of administering one or more doses of the checkpoint inhibitor to the subject prior to administration of the therapeutic CAR T-cell composition, wherein the subject does not receive any further doses of the checkpoint inhibitor after administration of the therapeutic CAR T-cell composition.

18. A checkpoint inhibitor for use in a method for treating cancer in a subject which method comprises the following steps:
(i) administering one or more doses of the checkpoint inhibitor to the subject; prior to
(ii) administering a therapeutic CAR T-cell composition to the subject
wherein the subject does not receive any further doses of the checkpoint inhibitor after administration of the therapeutic CAR T-cell composition.

19. The use of a checkpoint inhibitor in the manufacture of a medicament for preconditioning a subject who is about to receive a therapeutic CAR T-cell composition, which preconditioning method comprises the step of administering one or more doses of the checkpoint inhibitor to the subject prior to administration of the therapeutic CAR T-cell composition, wherein the subject does not receive any further doses of the checkpoint inhibitor after administration of the therapeutic CAR T-cell composition.

20. The use of a checkpoint inhibitor in the manufacture of a medicament for treating cancer in a subject, which method comprises the following steps:
(i) administering one or more doses of the checkpoint inhibitor to the subject; prior to
(ii) administering a therapeutic CAR T-cell composition to the subject
wherein the subject does not receive any further doses of the checkpoint inhibitor after administration of the therapeutic CAR T-cell composition.
